(19) United States
(12) Patent Application Publication
(10) Pub. No.: US 2012/0310311 A1
(43) Pub. Date: Dec. 6, 2012

(54) Body Contouring Apparatus

(75) Inventor: Itzhak Elkah, Irvine, CA (US)

(73) Assignee: SYNERON MEDICAL LTD., Yoqneam,Illit (IL)

(21) Appl. No.: 13/522,501

(22) PCT Filed: Feb. 20, 2011
(86) PCT No.: PCT/IL11/00170

§ 371 Date: Aug. 14, 2012

Related U.S. Application Data
(60) Provisional application No. 61/307,517, filed on Feb. 24, 2010.

(51) Int. Cl. A61N 5/00; A61F 7/08

(52) U.S. Cl. 607/102; 607/101; 607/112

(57) ABSTRACT

The current method and apparatus provide a body contouring apparatus employed in cosmetic body contouring treatments such as, but not limited to, fat reduction, body circumference reduction, cellulite reduction, skin tightening and skin rejuvenation at a clinical or dedicated professional setting, in the privacy of the subject's own home or at a comfortable and relaxing fitness room, sports activity center or spa-like environment.

### TECHNICAL FIELD

The method and apparatus relate to the field of cosmetic body contouring devices.

### BACKGROUND

Cosmetic body shaping treatments commonly involve employment of complex devices by professionals in clinical settings. The numerous methods of treatments apply various forms of energy to the tissue, primarily consisting of heating energy, in a form of light, radiofrequency (RF), ultrasound and microwaves and any combination thereof.

These methods commonly involve employing an applicator to house and translate a source of application energy over the skin, following various patterns of translation paths The translation of the applicator is often mechanical and carried out manually or automatically. Moreover, manual translation of the applicator is commonly subject to deviations from the predetermined treatment protocol path pattern and may bring about non-uniform treatment of the tissue.

The tissues to be treated commonly include the skin and various skin layers such as collagen tissue, hair and hair growth components such as hair follicles and sub-cutaneous fat tissue.

Apparatuses such as that disclosed in U.S. Pat. No. 6,470,216 are complex, often mobile, apparatuses employed in a clinical setting as alternatives to plastic surgery. The apparatus disclosed in U.S. Pat. No. 6,470,216 includes a hand held conforming template that is mobile and houses energy delivery devices delivering heating energy to the body structure to be treated primarily to effect remodeling of the collagen tissue.

US Patent Application Publication No. 2008/0183251 also discloses a device and method for remodeling of the collagen tissue employing primarily tri-polar RF energy by translating an applicator over the tissue to be treated.

US Patent Application Publication No. 2007/0239077 disclosed a cosmetic and/or medical apparatus in supervised clinical setting employing ultrasound energy for lipolysis and body contouring. This application also discloses an electromechanical device that moves at least a portion of the acoustic elements to different locations on the cuff.

### BRIEF SUMMARY

The current method and apparatus provide a body contouring apparatus employed in cosmetic body contouring treatments such as, but not limited to, fat reduction, body circumference reduction, cellulite reduction, skin tightening and skin rejuvenation at a clinical or dedicated professional setting, in the privacy of the subject's own home or at a comfortable and relaxing fitness room, sports activity center or spa-like environment.

In accordance with an exemplary embodiment of the current method and apparatus, the apparatus may include an optionally disposable, removably attached, energy delivery modular component that may be attached to a body accommodating surface connected to any suitable commonly available or dedicated body treatment accessory, converting it into a cosmetic body contouring apparatus. Such an accessory may include and/or be associated with, for example, a body massaging unit such as a massage recliner, a portable vibration applying cushion, a body support accessory such as a human-back supporting belt, sports clothing, a waist-trimming massage belt or treadmill. The energy delivery modular component feature makes the body contouring apparatus readily portable and convenient for use in a non-clinical setting.

In another embodiment of the current method and apparatus, the RF treatment applied by an array of energy delivery modular components results in an effect of progression of a heated linear zone in a tissue layer progressing along a predetermined path in accordance with a treatment protocol. The linear and/or rotational sweeping tissue heating wave effect is created without physical or mechanical movement of, for example, an applicator, a cuff, a body accommodating surface and/or electrodes. The linear and/or rotational sweeping tissue heating wave effect also provides a soothing and relaxing sensation of a sweeping wave of warmth across and area of tissue to be treated.

In accordance with another exemplary embodiment of the current method and apparatus the apparatus may also include safety features such as one or more sensors, and a controller including a timer and a time control unit so that to automatically monitor and control employment of the apparatus by a user in a private non-clinical setting.

In yet another exemplary embodiment of the current method and apparatus, the apparatus may also be designed to apply peripheral and circumferential cosmetic treatment to temporarily increase muscle tone, blood-circulation and fat cell metabolism to contour circumferential areas such as, but not limited to, the waist area and proximal limbs.

In still another exemplary embodiment of the current method and apparatus, the apparatus may apply invasive and/or non-invasive types of cosmetic treatment to the body including forms of energy such as, but not limited to RF energy, light energy and mechanical energy.

In another exemplary embodiment of the current method and apparatus the energy delivery modular component may be supplied by a rechargeable and/or disposable power source.

### BRIEF DESCRIPTION OF THE DRAWINGS

The present method and apparatus will be understood and appreciated from the following detailed description, taken in conjunction with the drawings in which:

FIGS. 1A and 1B are simplified views of exemplary embodiments of the current method and apparatus;

FIGS. 2A, 2B and 2C are simplified views of three configurations of the exemplary embodiment of FIG. 1B;

FIGS. 3A, 3B, 3C, 3D and 3E are simplified plan view and side view illustrations of exemplary embodiments and electrode configurations of an energy delivery modular component in accordance with the current method and apparatus;

FIG. 4 is a simplified view of a user interface in accordance with another exemplary embodiment of the current method and apparatus;

FIGS. 5A, 5B, 5C, 5D and 5E are simplified plan view and cross-sectional view illustrations of generation of a linear sweeping heating wave effect in accordance with an exemplary embodiment of the current method and apparatus;

FIGS. 6A-6J are simplified plan view illustrations of several configurations of the linear sweeping wave effect of FIGS. 5A-5E in accordance with the current method and apparatus;

FIGS. 7A-7E are simplified plan view illustrations of generation of a peripheral rotational heating wave effect in accordance with an exemplary embodiment of the current method and apparatus;

FIGS. 8A-8C are simplified plan view illustrations of generation of a linear sweeping heating wave effect across an array of RF electrodes of an energy delivery modular component in accordance with an exemplary embodiment of the current method and apparatus;

FIGS. 9A-9D are simplified illustrations of a linear sweeping tissue heating wave effect generated by an array of energy delivery modular components in accordance with an exemplary embodiment of the current method and apparatus; and

FIGS. 10A-10E are simplified plan view illustrations of several configurations of the linear sweeping wave effect across an array of energy delivery modular components in accordance with the current method and apparatus.

### DETAILED DESCRIPTION OF THE CURRENT METHOD AND APPARATUS

For the purpose of this disclosure the term “Cuff” as used hereinbelow means any belt-like band operative to partially or fully enclose the circumference of a body part such as a limb or torso.

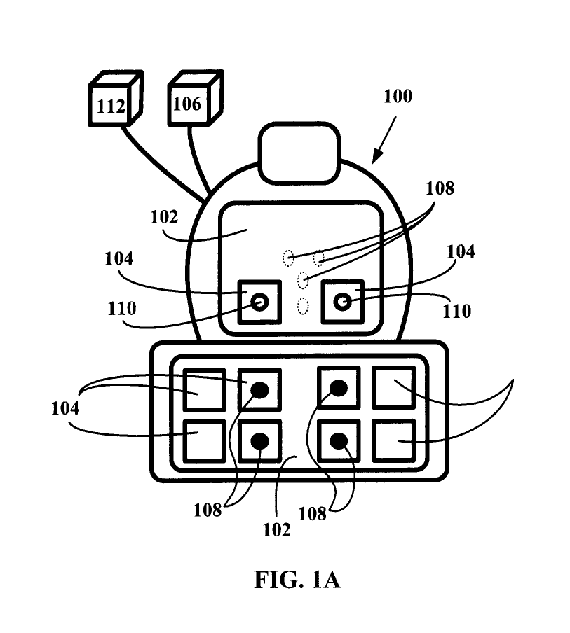

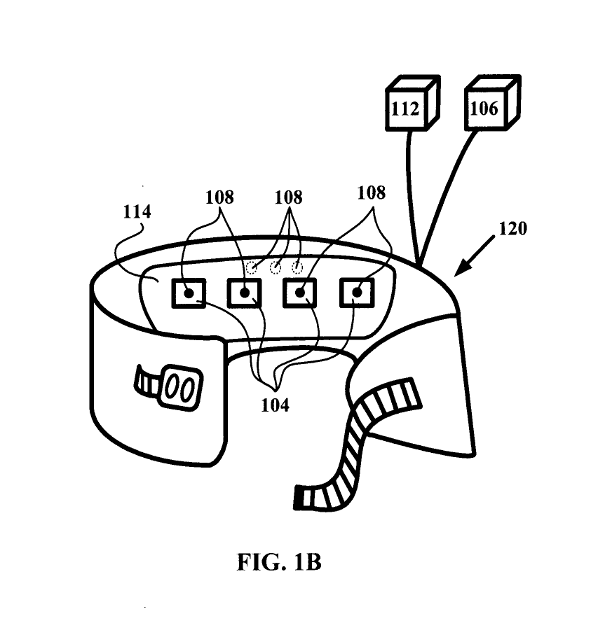

Referring now to FIGS. 1A and 1B, which are simplified views of exemplary embodiments of the current method and apparatus. FIG. 1A illustrates a seat 100 having a body accommodating surface 102 and one or more energy delivery modular components 104 integrally attached thereto. Alternatively, body accommodating surface 102 may be removably attached, independently or together with one or more energy delivery modular components 104 to a piece of furniture such as a massage recliner employing a removable attachment system (For example, 3M Scotch® Hook-and-Loop Tape) or attachment clips.

Energy delivery modular components 104 may be replaceable and/or disposable and attach to body accommodating surface 102 with detachable connectors such as ECG snap type connectors, miniature coaxial (MC) type RF Connectors or any other suitable detachable connector.

Body accommodating surface 102 may include or connect to a power source 106 communicating with modular components 104. Power source 106 may be and/or include a source of RF energy. Optionally and alternatively, each modular component 104 may be independently connected to a source of power (not shown) that may be and/or include a source of RF energy. The sources of power supplying modular components 104 may be rechargeable or disposable.

Body accommodating surface 102 may also include a disposable fenestrated sheet (Not shown), made of a thin disposable material such as paper, fabric and/or textile or a thin plastic sheet, to be replaced following each treatment session. The fenestrations of the disposable fenestrated sheet may overlap the locations of energy delivery modular components 104 to enable contact thereof with the surface of the body part to be treated.

Energy delivery modular components 104 may be operative to apply minimally invasive and/or non-invasive types of cosmetic treatment to the body including applying forms of energy such as, but not limited to, RF energy, light energy and mechanical energy as will be explained in detail hereinbelow.

Optionally, energy delivery modular components 104 may also include one or more mechanical energy delivery elements 108 to deliver vibration energy to tissue to be treated. Mechanical energy elements 108 may include, for example, vibrating balls that temporarily increase muscle tone, blood circulation and fat tissue metabolism in the local area to be treated.

Optionally, body accommodating surface 102 may also include one or more mechanical energy delivery elements 108 to deliver vibration energy to tissue to be treated.

Modular component 104 may be connected to a controller 112 and communicate therewith. Controller 112 may include a timer and a time control and counter unit and be operative to control modular components 104, body accommodating surface 102 and power source 106.

Modular component 104 may also include one or more sensors 110 selected from a group consisting of a temperature sensor, a pressure sensor and an impedance sensor. Optionally, body accommodating surface 102 may also include one or more sensors 110.

Sensors 110 provide controller 112 with feedback consisting of one or more treatment parameters selected from a group consisting of temperature, pressure, and tissue impedance. Controller 112 timer provides controller 112 with time information input. Controller 112 may also be operative to activate or reset the timer in accordance with a predetermined cosmetic treatment protocol.

Controller 112, based on treatment parameters received from sensors 110, time information received from the timer and in accordance with a predetermined cosmetic treatment protocol is operative to carry out one or more of the following functionalities: adjust cosmetic treatment parameters such as RF voltage and frequency, cease and resume treatment application and alert a user regarding information relating to treatment parameters and duration of treatment.

Controller 112 time control and counter unit is operative to receive time information input from the timer and an RFID located in modular component 104 and/or body accommodating surface 102 to monitor and control time associated functionalities in accordance with a predetermined treatment protocol. Such functionalities include limiting duration of treatment to avoid overheating of tissue, limiting use time and/or providing a disposable modular component 104 replacement time alert following a predetermined number of uses or maximal allowable duration of use, and providing controller 112 with usage data such as number of uses, duration of use and dates of use. Controller 112 is operative to store the information received from time control and counter unit in a database.

Controller 112 is also operative to remotely communicate and receive information from the Internet relating to cosmetic treatment protocols and treatment parameter settings and adjust cosmetic treatment parameters in accordance with the received information.

FIG. 1B illustrates another exemplary embodiment of the current method and apparatus, in which body accommodating surface 114 is integrally attached to a portion of a waist cuff 120. Alternatively, body accommodating surface 114 may be removably attached to waist cuff 120 employing a removable attachment system layer (For example, 3M Scotch® Hook-and-Loop Tape) or attachment clips. Modular component 104 may be connected to controller 112 and communicate therewith as explained hereinabove.

Optionally, one or more mechanical energy elements 108 may also be located in one or more modular components 104 and/or body accommodating surface 114 of waist cuff 120. Waist cuff 120 may also include a power source 106 to supply modular components 104. The power source may be a standalone unit/module, or be carried in a pouch attached to the cuff or a limb of a person using the cuff.

Energy delivery modular component 104 may, independently or in association with cuff 120 and body accommodating surface 102, constitute a portable body contouring apparatus, carried in, for example, a suitable carrying case, operative to be employed in a non-clinical environment and optionally in association with commonly used home, spa and fitness room furniture and accessories such as fitness trainer machines and treadmills.

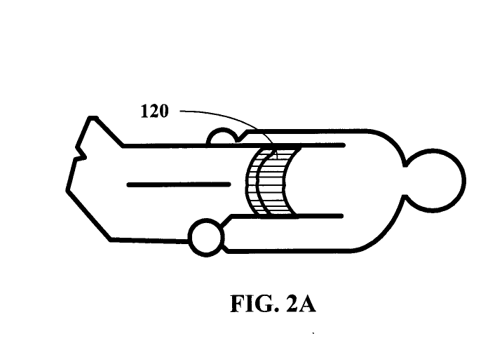

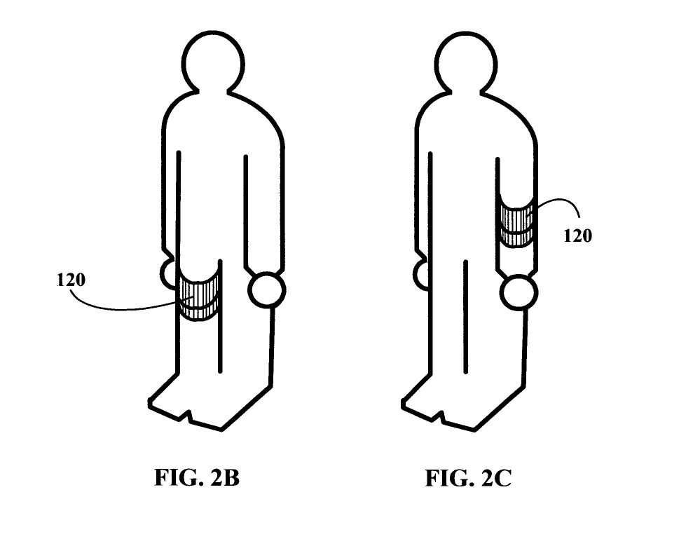

Referring now to FIGS. 2A, 2B and 2C, which are simplified views of three application configurations of the exemplary embodiment of FIG. 1B. FIG. 2A illustrates employment of waist cuff 120 in cosmetic circumferential treatment of the waist and abdomen area. Cosmetic treatment of this area may contribute to fat reduction, waist area circumference reduction, cellulite reduction, skin tightening and skin rejuvenation.

Cuff 120 may also be adjusted to be employed in an application configuration for the cosmetic circumferential treatment of the proximal portion of a limb such as the proximal portion of the leg, as shown in FIG. 2B and/or the proximal portion of the arm, as shown in FIG. 2C, to contribute to fat reduction, limb circumference reduction and sagging skin treatment such as skin tightening and skin rejuvenation.

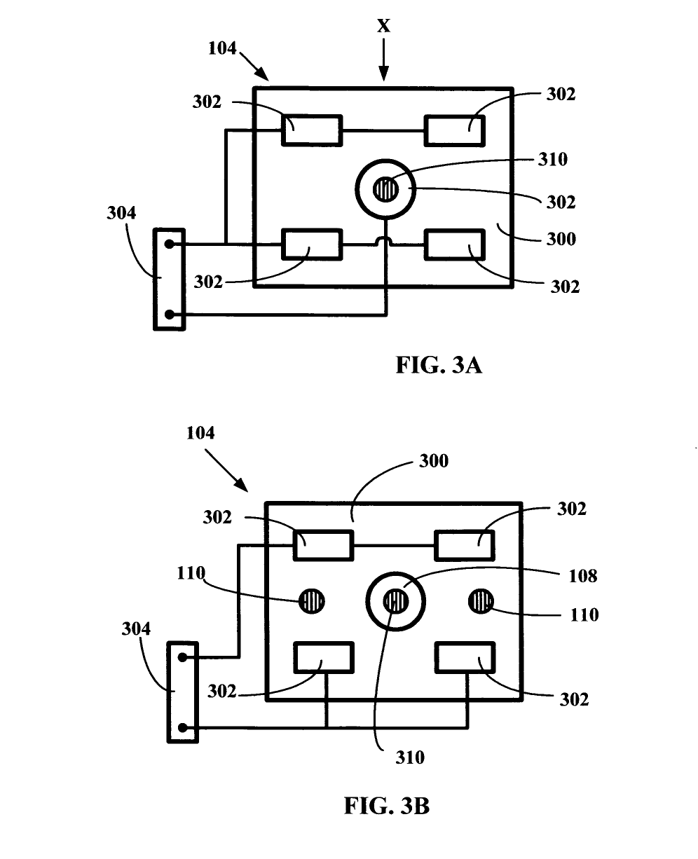

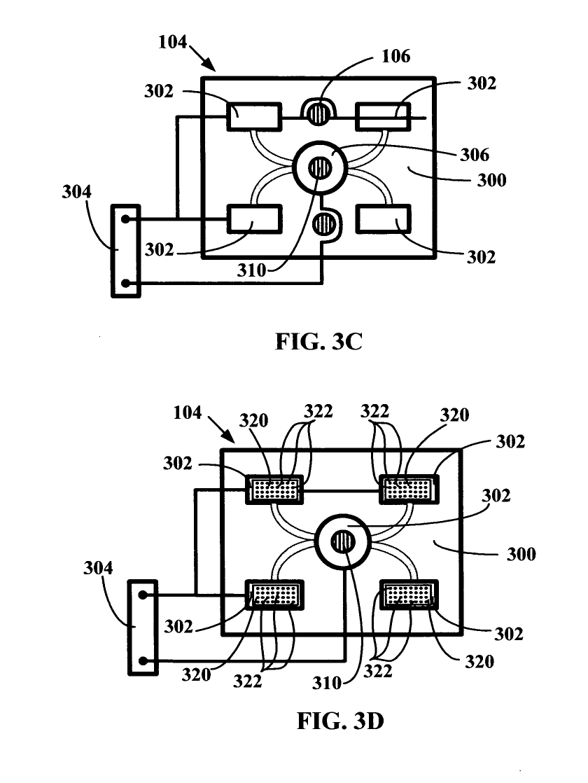

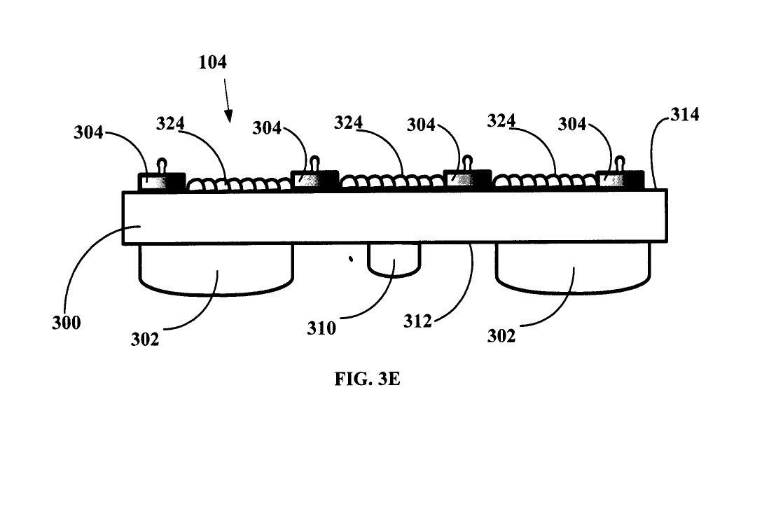

Reference is now made to FIGS. 3A, 3B, 3C, 3D and 3E, which are simplified plan view and cross-sectional view illustrations of exemplary embodiments and electrode configurations of an energy delivery modular component 104 in accordance with the current method and apparatus. As shown in FIG. 3A, energy delivery modular component 104 includes a carrier 300 and one or more RF electrodes 302 attached thereto. Carrier 300 may be made of a rigid, semi-rigid or flexible material and may be padded with a soft fabric. RF electrodes 302 communicate with controller 112 (FIG. 1) and are supplied by power source 106 connected to modular component 104 via a connector 304. Alternatively, energy delivery modular component 104 may be connected to power source 106 indirectly through body accommodating surface 102 via a connector 304.

Modular component 104 may also include one or more sensors 110 selected from a group consisting of a temperature sensor, a pressure sensor and an impedance sensor operative to communicate with controller 112 (FIG. 1). Controller 112 is operative to store information received from sensors 110 in the database and control RF electrodes 302. Carrier 300 may also include a rigid or flexible printed circuit board (PCB) to house all electrical communication wires between modular component 104 elements such as RF electrodes 302, connector 304 and sensors 106.

Energy delivery modular component 104 and/or body accommodating surface 102 may also include user identification capability such as RFID (not shown) to control and limit usage thereof. For example, the use of a disposable component may be recorded in the RFID in a way that it will prevent repeat use of the same disposable component.

RF treatment energy may be applied in a monopolar, bi-polar and multipolar mode and electrodes 302 would be constructed accordingly. RF electrodes 302 may also be operative to apply RF energy in pulse or continuous form at a power level in the range, but not limited to, between 1 W and 40 W. Commonly, the lower RF power settings are applied in non-clinical cosmetic treatment applications whereas the higher RF power settings are applied in professionally supervised or clinical cosmetic treatment applications. For example, RF power settings for non-clinical cosmetic treatment applications may commonly be in the range, but not limited to, between 1 W-20 W and more commonly in the range, but not limited to, between 7 W-15 W. RF power settings for professional supervised or clinical cosmetic treatment applications may commonly be in the range, but not limited to, between 18 W-40 W and more commonly in the range, but not limited to, between 25 W-35 W. The generated RF energy by electrodes 302 is employed to apply one or more cosmetic treatments selected from a group consisting of fat reduction, body circumference reduction, cellulite reduction, skin tightening and skin rejuvenation as well as to increase blood-circulation and fat cell metabolism to contour the body.

The configuration shown in FIG. 3A is a multipolar configuration of electrodes 302 of modular component 104. Temperature sensor 310 in this configuration may be a thermistor operative to monitor the tissue temperature during application of the RF cosmetic treatment. Alternatively, sensor 310 may be a pressure sensor operative to communicate with controller 112 and convey thereto information relating to contact or lack thereof between modular component 104 electrodes 302 and surface of tissue to be treated. Controller 112 may activate or deactivate one or more modular component 104 electrodes 302 or adjust cosmetic treatment parameters in accordance with the input from sensor 110.

In accordance with another exemplary embodiment of the current method and apparatus and as shown in FIG. 3B, modular component 104 may include a bipolar configuration of electrodes 302. A temperature sensor 110 such as a thermistor may be located between each pair of electrodes 302 operative to monitor the tissue temperature during application of the RF cosmetic treatment. In this embodiment, modular component 104 also includes one or more mechanical energy delivery elements 108 and a sensor 310 to monitor contact or lack thereof between modular component 104 electrodes 302 and surface of tissue to be treated as described hereinabove.

Referring now to FIG. 3C, which is another exemplary embodiment of energy delivery modular component 104 in accordance with the current method and apparatus. In this embodiment, carrier 300 also includes a light emitting element 306 operative to emit light in the visible and Infra Red (IR) range to heat a segment of tissue to be treated. In this embodiment, sensor 310 may be a thermistor. Both light source 306 and sensor 310 are operative to communicate with controller 112 and power source 106. Optionally, light emitting element 306 may be an LED type light source.

In accordance with yet another exemplary embodiment of the current method and apparatus, and as shown in FIG. 3D, RF electrodes 302 may also include one or more applying elements carriers 320 having on one surface thereof a plurality of RF voltage applying elements 322 in a spaced apart pattern as described in assignee's PCT Application Publication No. WO2009/072108, the disclosure of which is hereby incorporated by reference. Alternatively, applying elements 322 may be light emitting elements such as LEDs, VCSELs, laser diodes, and other.

In accordance with still another exemplary embodiment of the current method and apparatus, RF electrodes 302 may include a plurality of spaced apart protruding conducting elements (not shown) configured to contact a skin surface at a plurality of discrete and separate locations and penetrate the layers of the skin such as, for example, the epidermis, as described in assignee's U.S. Patent Application 2006/0047281 the disclosure of which are hereby incorporated by reference.

Reference is now made to FIG. 3E, which is a simplified side view illustration of another exemplary embodiment of the energy delivery modular component 104 of FIG. 3A viewed from the direction indicated by arrow X. Carrier 300 includes one or more electrodes 302 attached to an energy delivery surface 312. An attachment surface 314, opposite to surface 312, includes one or more ECG snap type connectors 304 readily connectable to corresponding ECG snap type connector receptacles on body accommodating surface 102. Connectors 304 Attachment surface 314 may also be coated with a removable attachment system layer 324 (For example, 3M Scotch® Hook-and-Loop Tape) or attachment clips. Modular component 104 also includes a sensor 310 attached to energy delivery surface 312 thereof operative to communicate with controller 112 (FIG. 1) via connectors 304.

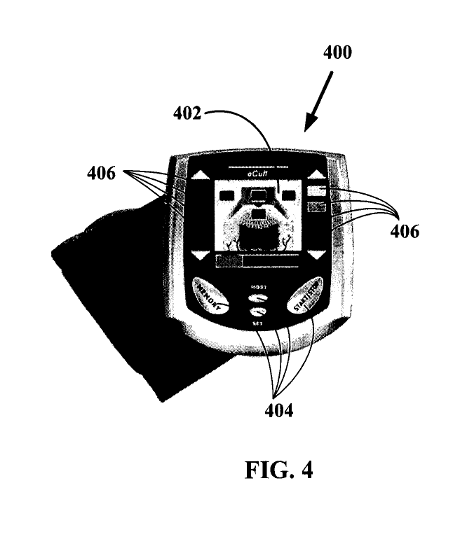

Reference is now made to FIG. 4, which illustrates a simplified view of a user interface 400 in accordance with another exemplary embodiment of the current method and apparatus. User interface 400 includes a monitor 402, which may be, for example, a touch screen monitor, action buttons 404 and indicators 406.

A user may employ interface 400 to select from a predetermined selection of treatment protocols and initiate or stop the treatment session.

User interface 400 monitor 402 may also present a user with information retrieved from controller 112 (FIG. 1) database such as treatment parameters, stored user information, preferences and settings and data received and stored from previous treatment sessions.

User interface may communicate with controller 112 directly via connector 304 or by wireless communication.

User interface 400 may also be operative to sound various auditory signals such as treatment beginning and end indications and alerts indicating deviation from treatment parameters. Generation of the audio signals may be controlled by controller 112 in accordance with a predetermined treatment protocol.

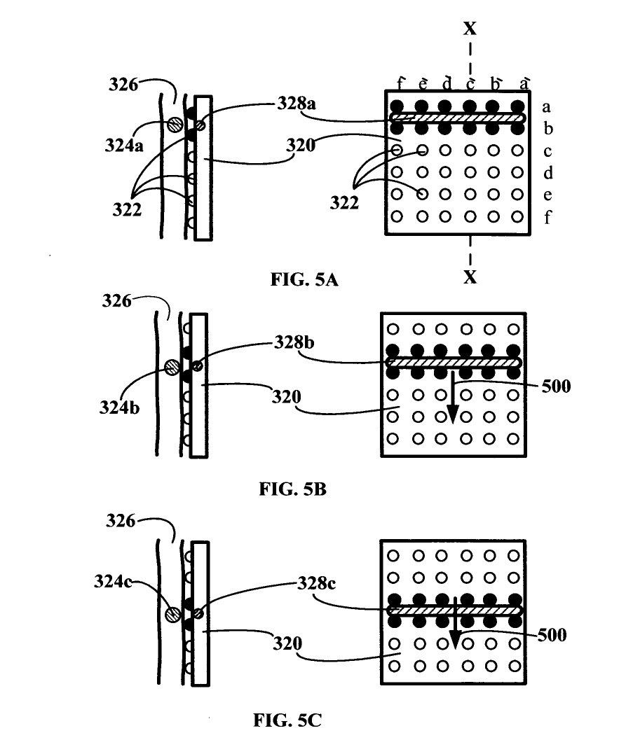

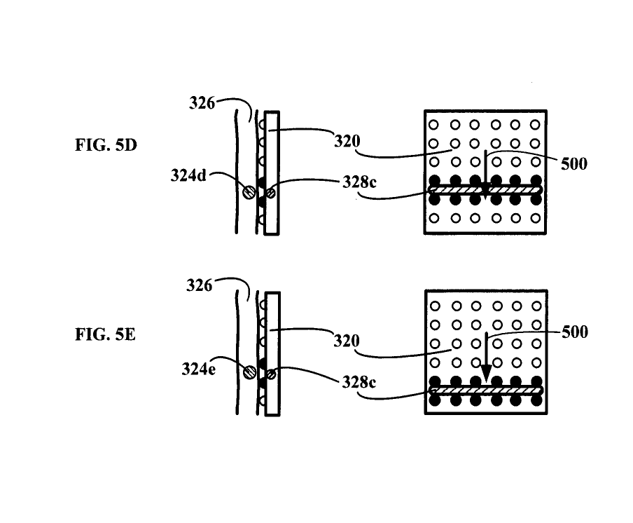

Referring now to FIGS. 5A-5E, which are simplified plan view and cross section view illustrations of RF electrode generation of a linear sweeping heating wave effect in accordance with an exemplary embodiment of the current method and apparatus. Each of FIGS. 5A, 5B, 5C, 5D and 5E is accompanied by a corresponding cross-sectional view taken along the axis X-X.

Electrode 302, of the type shown in FIG. 3D includes a carrier 320 and a plurality of RF voltage applying elements 322 in a spaced apart pattern. In FIG. 5A, elements 322 are arranged in an array along rows (a-f) and columns (a′-f′).

Controller 112 (FIG. 1) is operative to activate and deactivate individual elements 322 of RF electrode 304 in accordance with a predetermined treatment protocol.

As shown in FIG. 5A, only rows (a) and (b) are activated by controller 112, as indicated by the blackening of elements 322 of rows (a) and (b), and RF current flows from elements 322 in row (a), through tissue to be treated, to elements 322 in row (b) heating a linear zone 324a of tissue layer 326 therebetween and parallel thereto corresponding to an area 328a on carrier 300.

As illustrated in FIG. 5B, row (a) is now inactivated and only rows (b) and (c) are activated. RF current now flows from elements 322 in row (b), through tissue to be treated, to elements 322 in row (c). Heated linear zone 324a of tissue layer 326 has now cooled down and an adjacent linear zone 324b corresponding to an area 328b on carrier 300 is now heated.

In FIG. 5C, rows (a) and (b) are now inactivated and only rows (c) and (d) are activated. RF current now flows from elements 322 in row (c), through tissue to be treated, to elements 322 in row (d). Heated linear 324b zone of tissue layer 326 has now cooled down and an adjacent linear zone 324c corresponding to an area 328c on carrier 300 is now heated.

As shown in FIG. 5D, rows (a), (b) and (c) are now inactivated and only rows (d) and (e) are activated. RF current now flows from elements 322 in row (d), through tissue to be treated, to elements 322 in row (e). Heated linear 324c zone of tissue layer 326 has now cooled down and an adjacent linear zone 324d corresponding to an area 328d on carrier 300 is now heated.

In FIG. 5E, rows (a), (b), (c) and (d) are now inactivated and only rows (e) and (f) are activated. RF current now flows from elements 322 in row (e), through tissue to be treated, to elements 322 in row (f). Heated linear 324d zone of tissue layer 326 has now cooled down and an adjacent linear zone 324e corresponding to an area 328e on carrier 300 is now heated.

The RF treatment results in an effect of progression of heated linear zone 324 of tissue layer 326 progressing along a predetermined path in accordance with a treatment protocol in the direction indicated by the arrow assigned reference numeral 500 creating a linear sweeping tissue heating wave effect without physical or mechanical movement of, for example, cuff 120 or electrodes 302.

All directions of progression of the sweeping tissue heating wave effect described hereinbelow are given with respect to the drawing plane. It is appreciated that the apparatus may not be limited to any particular plane and may be operative in any orientation.

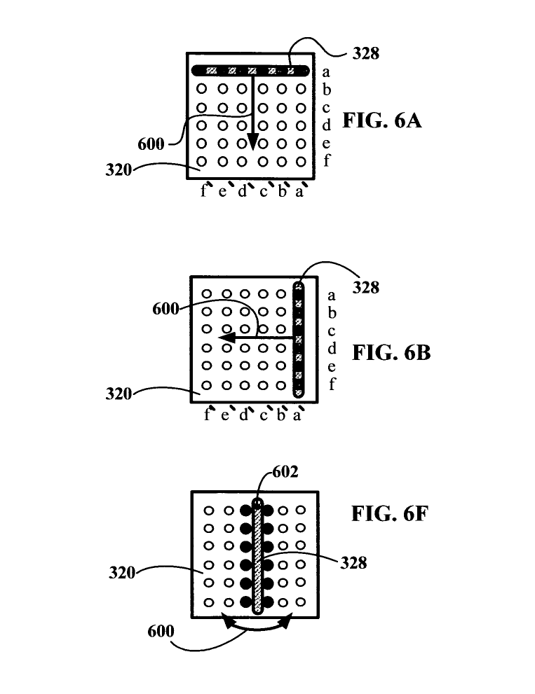

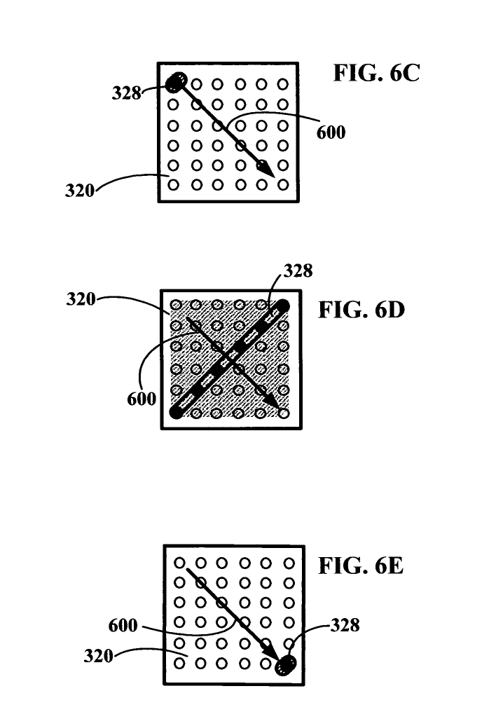

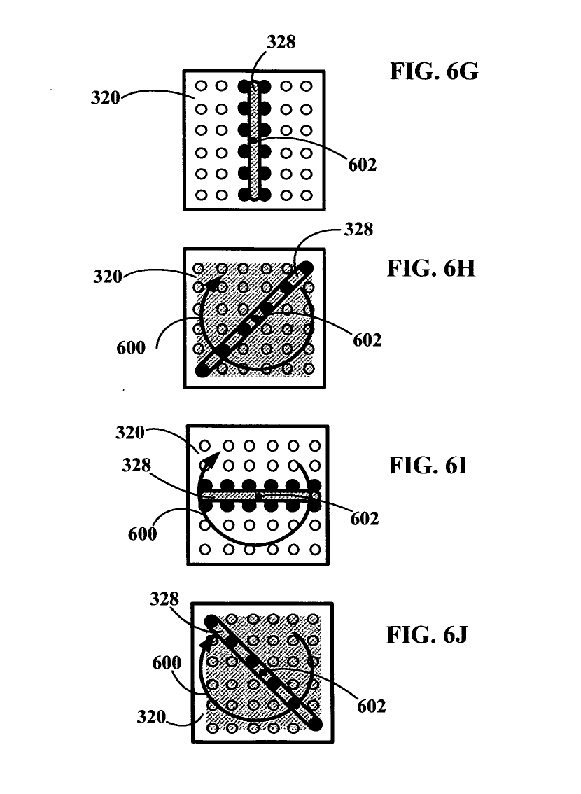

Turning now to FIGS. 6A to 6F illustrate several configurations of the linear sweeping wave effect of FIGS. 5A-5E in accordance with the current method and apparatus. FIG. 6A illustrates a horizontal linear sweeping tissue heating wave effect generated in a tissue layer (not shown) by a corresponding area 328 moving in a vertical direction from row (a) to row (f) in a direction indicated by arrow 600 and explained in detail hereinabove. This effect may progress repeatedly uni-directionally, for example, rows (a)-(f), (a)-(f), (a)-(f), etc., or, bi-directionally, for example, rows (a)-(f), (f)-(a), (a)-(f), (f)-(a), etc.

FIG. 6B illustrates a vertical linear sweeping tissue heating wave effect generated in a tissue layer (not shown) by a corresponding area 328 moving in a horizontal direction from column (a′) to column (f′) in a direction indicated by arrow 600. This effect may repeat uni-directionally, for example, columns (a′)-(f′), (a′)-(f′), (a′)-(f′), etc., or, bi-directionally, for example, columns (a′)-(f), (f′)-(a′), (a′)-(f′), (f′)-(a′), etc.

FIGS. 6C-6E illustrate a diagonal linear sweeping tissue heating wave effect generated in a tissue layer (not shown) by a corresponding area 328 moving in a diagonal direction from a corner W towards a corner Z in a direction indicated by arrow 600. This effect may repeat uni-directionally, for example, W to Z, W to Z, W to Z, etc., bi-directionally, for example, W to Z, Z to W, W to Z, Z to W, etc, or in a crisscross pattern, for example, between corners W, X, Y and Z: W to Z, X to Y, W to Z, X to Y, etc.

As shown in FIG. 6F a diagonal linear sweeping tissue heating wave effect generated in a tissue layer (not shown) by a corresponding area 328 may move in a pendulous pattern of progression pivoting around an imaginary point indicated by reference numeral 602 in a back and forth direction indicated by arrow 600.

As shown in FIGS. 6G-6J, RF electrode 302 may also generate a rotational heating sweeping wave effect, pivoting around an imaginary point indicated by reference numeral 602, generated in a tissue layer (not shown) by a corresponding area 328 in a in circular direction indicated by arrow 600. FIGS. 6G, 6H, 6I and 6J illustrate such a rotational heating sweeping wave effect at four selected exemplary stages of such a rotational effect. The sweeping wave effect enables heat application to a tissue segment substantially larger than by conventional electrodes.

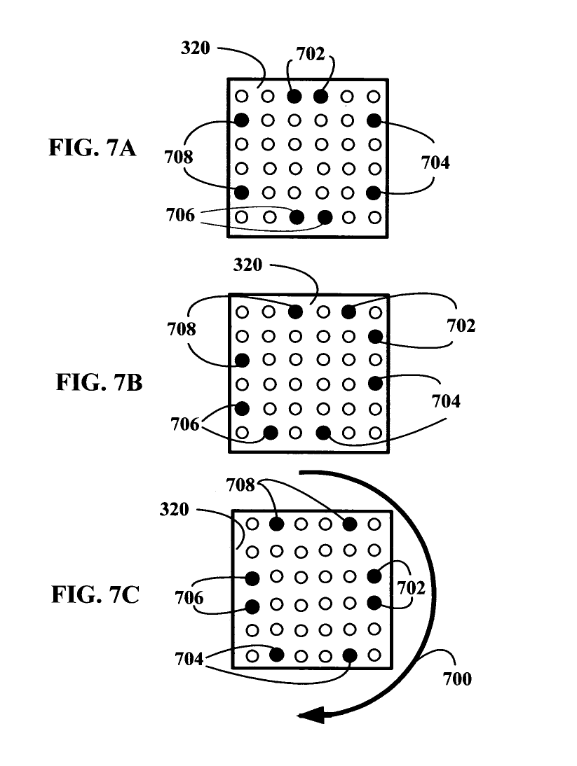

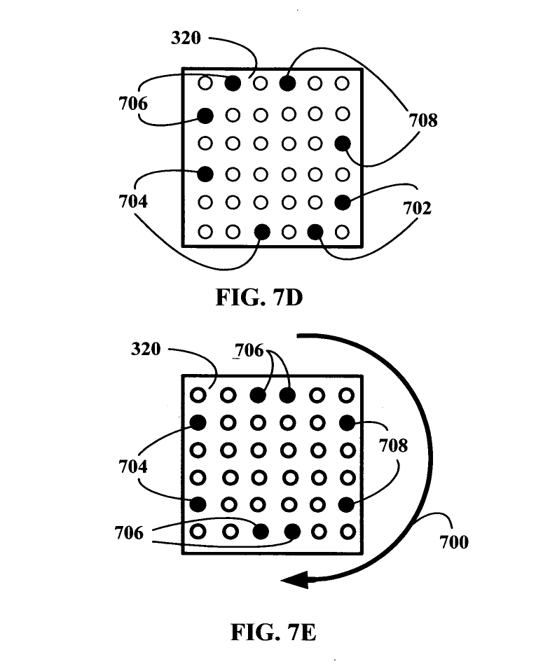

Turning now to FIGS. 7A-7E, which are simplified plan view illustrations of RF electrode 302 generation of a peripheral rotational heating wave effect in accordance with an exemplary embodiment of the current method and apparatus. Electrode 302, of the type shown in FIG. 3D includes a carrier 320 and a plurality of RF voltage applying elements 322 in a spaced apart pattern. As shown in FIG. 7A, only four pairs 702, 704, 706 and 708 of elements 322 along the outer periphery, for example, rows (a) and (f), and columns (a′) and (f′) are activated by controller 112 (FIG. 1) as indicated by the blackening thereof.

FIG. 7B illustrates the shifting of the position of each of pairs 702, 704, 706 and 708 along the periphery in a clockwise direction.

FIG. 7C illustrates another shifting of the position of each of pairs 702, 704, 706 and 708 along the periphery in a clockwise direction as indicated by arrow 700.

FIG. 7D illustrates yet another shifting of the position of each of pairs 702, 704, 706 and 708 along the periphery in a clockwise direction.

At the stage shown in FIG. 7E the continuous shifting results in a 180 degree peripheral rotation of a tissue heating wave in a clockwise direction as indicated by arrow 700.

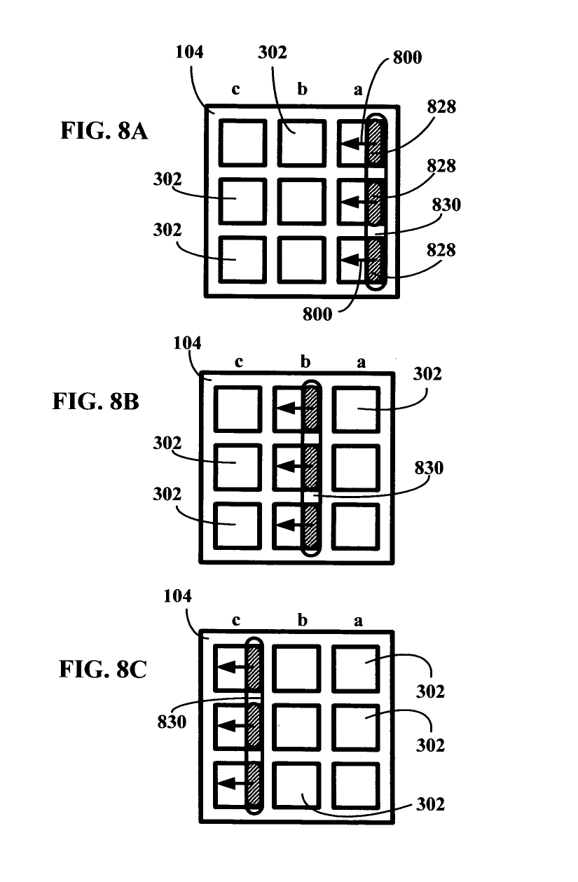

Referring now to FIGS. 8A-8C which are plan view illustrations of generation of a linear sweeping heating wave effect by RF electrodes 302 of the type shown in FIG. 3D across energy delivery modular component 104 in accordance with an exemplary embodiment of the current method and apparatus. In FIGS. 8A-8C nine electrodes 302 are arranged in an array of three rows and three columns (a), (b) and (c). In FIG. 8A, only electrodes 302 of components 104 in column (a) are activated generating three vertical linear sweeping tissue heating wave effects generated in a tissue layer (not shown) by corresponding areas 828. It will be appreciated that the areas in the tissue corresponding to areas 828 may expand radially due to heat convection and adjacent heated areas may coalesce to create a linear heated tissue area corresponding to area 830. Activating components 104 electrodes 302 as described in detail hereinabove, may create a vertical linear sweeping tissue heating wave effect 830 moving in a horizontal direction from column (a) to column (c) in a direction indicated by arrows 800.

In FIG. 8B, components 104 of column (a) are inactivated and only electrodes 302 of components 104 in column (a) are activated. In FIG. 8C, components 104 of column (b) are inactivated and only electrodes 302 of components 104 in column (c) are activated. This serial process generates a vertical linear sweeping tissue heating wave effect generated in a tissue layer (not shown) by a corresponding area 828 moving in a horizontal direction from column (a) to column (c) in a direction indicated by arrows 800.

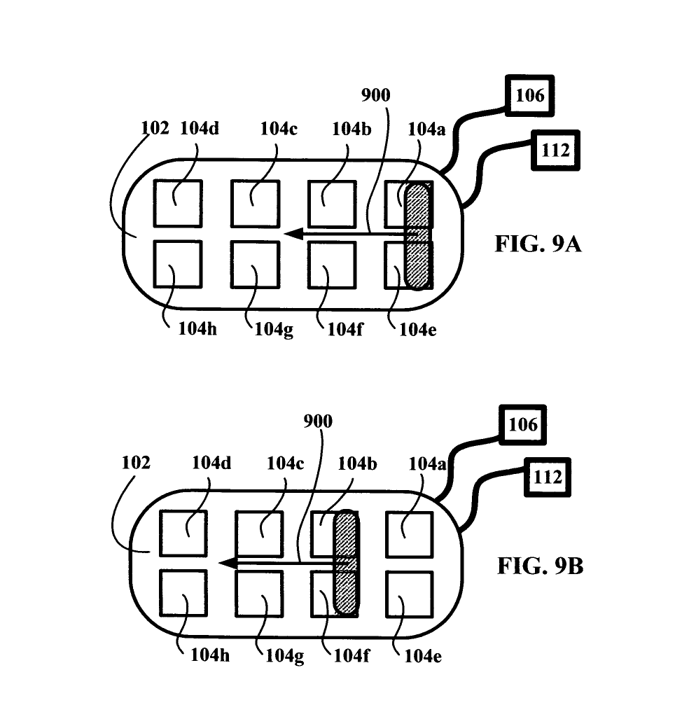

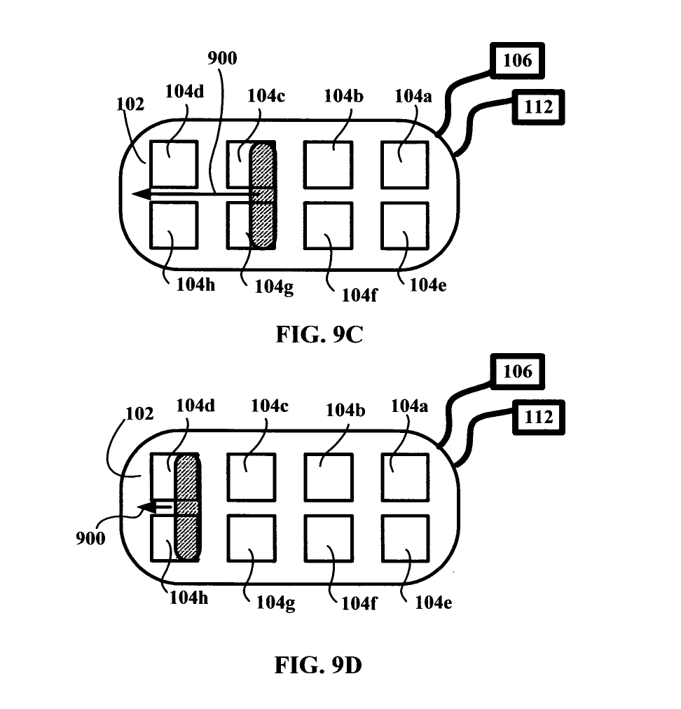

Referring now to FIGS. 9A-9D, which are simplified illustrations of a linear sweeping tissue heating wave effect generated by an array of energy delivery modular components 104 of the type shown in FIG. 3D in accordance with an exemplary embodiment of the current method and apparatus. As shown in FIG. 9A, a multiplicity of energy delivery modular components 104 are attached to a body accommodating surface 102. As described hereinabove, energy delivery modular components 104 are connected to, and supplied by power source 106 and controlled by a controller 112. Energy delivery modular components 104a, 104b, 104c, 104d, 104e, 104f, 104g and 104h are arranged in a double-line array.

In FIG. 9A only modular components 104a and 104e are activated heating a linear zone of tissue in contact therewith corresponding to an area 930 on modular components 104a and 104e, in a linear fashion as described in detail hereinabove and in a direction indicated by arrow 900.

As shown in FIG. 9B, components 104a and 104e are now inactivated and only components 104b and 104f are activated heating the tissue in contact therewith in a linear fashion as described hereinabove in a direction indicated by arrow 900.

In FIG. 9C, components 104b and 104f are now inactivated and only components 104c and 104g are activated heating the tissue in contact therewith in a linear fashion as described hereinabove in a direction indicated by arrow 900.

In FIG. 9D, components 104c and 104g are now inactivated and only components 104d and 104h are activated heating the tissue in contact therewith in a linear fashion as described hereinabove in a direction indicated by arrow 900.

This results in a vertical linear sweeping tissue heating wave effect moving in a horizontal direction along tissue to be treated in a direction indicated by arrows 900. It will be appreciated that the vertical linear sweeping tissue heating wave effect may also be generated by modular components of any of the types described hereinabove.

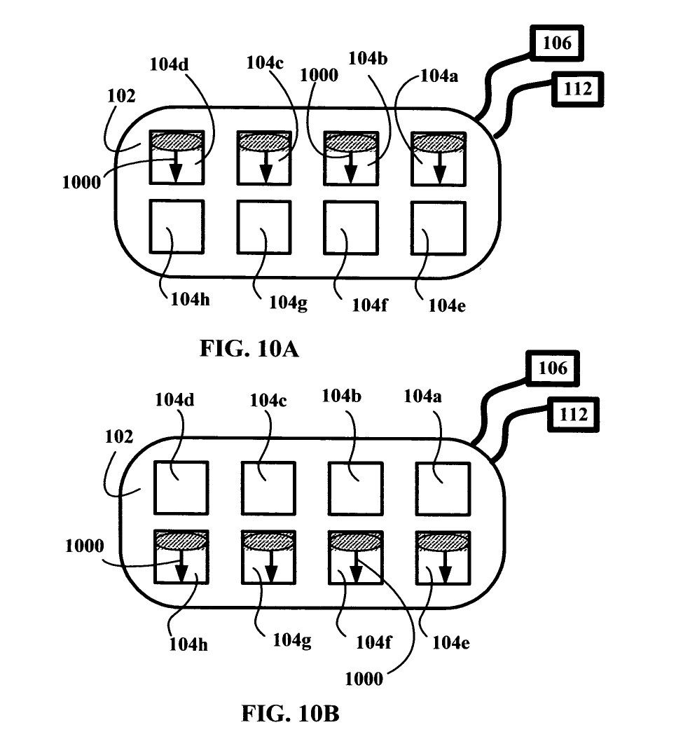

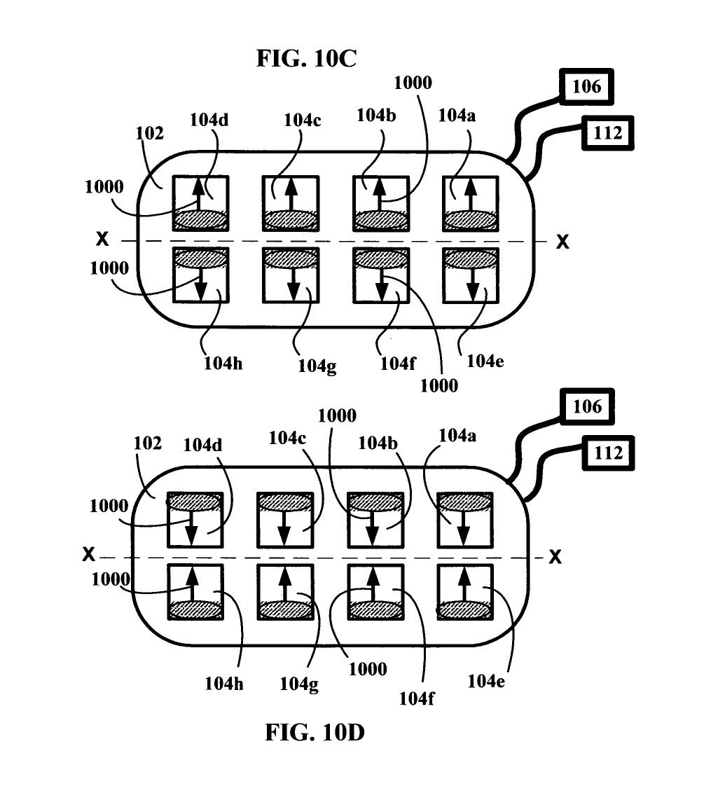

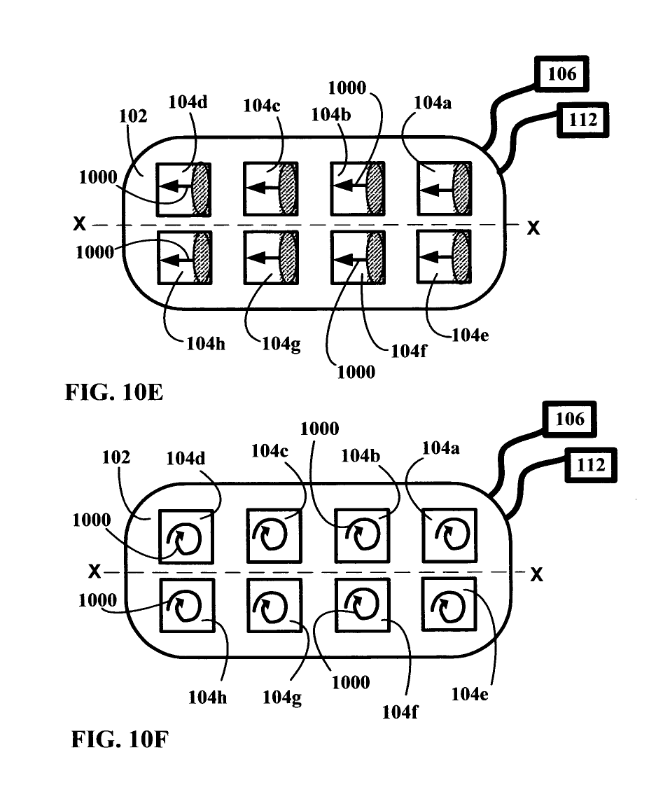

Referring now to FIGS. 10A-10E, which illustrate multiple configurations of linear and rotational sweeping tissue heating wave effects generated by an array of energy delivery modular components 104 attached to a body accommodating surface 102 in accordance with an exemplary embodiment of the current method and apparatus. FIGS. 10A and 10B illustrate a horizontal linear sweeping tissue heating wave effect moving in a vertical direction from components 104a, 104b, 104c and 104d towards corresponding components 104e, 104f, 104g and 104h. The horizontal linear sweeping tissue heating wave effect may repeat uni-directionally or bi-directionally as described in detail in FIG. 6A hereinabove.

As shown in FIG. 10C, two horizontal linear sweeping tissue heating wave effects may move in a vertical direction away from an imaginary central axis indicated by a broken line X. FIG. 10D illustrates two horizontal linear sweeping tissue heating wave effects that may move in a vertical direction towards an imaginary central axis indicated by a broken line X. FIG. 10E illustrates a linear sweeping tissue heating wave effect configuration progressing in a clockwise direction along the periphery of body accommodating surface 102.

Any one of the aforementioned configurations may also employ rotational sweeping tissue heating wave effects described hereinabove and shown in FIG. 10F. All of the configurations described hereinabove may be controlled by controller 112 in accordance with a predetermined treatment protocol, input of treatment parameters from sensors 110, the timer and time control and counter unit and/or be configured manually employing interface 400.

The RF treatment described hereinabove results in an effect of progression of heated linear zone of a tissue layer progressing along a predetermined path in accordance with a treatment protocol in the direction indicated by the arrows assigned reference numeral 1000 creating a linear and rotational sweeping tissue heating wave effect heating large tissue segments without physical or mechanical movement of, for example, cuff 120, body accommodating surface 102 and/or electrodes 302.

It will be appreciated by persons skilled in the art that the present method and apparatus are not limited to what has been particularly shown and described hereinabove. Rather, the scope of the invention includes both combinations and sub-combinations of various features described hereinabove as well as modifications and variations thereof which would occur to a person skilled in the art upon reading the foregoing description and which are not in the prior art.

1. A body contouring apparatus comprising:
at least one body accommodating surface operative to accommodate at least part of a subject's body;
a controller;
a source of RF energy; and
at least one energy delivery modular component removably connected to said body accommodating surface and having at least two RF delivery electrodes, operative to communicate with said controller and said source of RF energy and generate a sweeping tissue heating wave effect creating a progression of a heated zone across a segment of tissue to be treated.

2. The apparatus according to claim 1, wherein said apparatus also comprising:
at least one sensor selected from a group consisting of a temperature sensor, a pressure sensor, an impedance sensor;
a timer; and
wherein said controller operative to communicate with said sensor and timer, store in a database data received therefrom and control and monitor treatment parameters related to said energy delivery modular component in accordance with a predetermined treatment protocol.

3. The apparatus according to claim 1, wherein at least one of said body accommodating surface and energy delivery modular component is stationary.

4. The apparatus according to claim 1, wherein said effect is at least one of linear and rotational sweeping tissue heating wave effect.

5. The apparatus according to claim 1, wherein said energy delivery modular component also includes at least one light emitting element operative to emit light in at least one of visible and Infra Red range to heat a segment of tissue to be treated.

6. The apparatus according to claim 2, wherein said treatment parameters are at least one parameter selected from a group consisting of temperature, pressure and RF voltage and frequency.

7. The apparatus according to claim 1, wherein said body accommodating surface and/or said energy delivery modular component are operative to apply mechanical energy to said part of said subject's body in contact therewith and wherein said controller is also operative to monitor and control said applied mechanical energy.

8. The apparatus according to claim 1, wherein said body accommodating surface is associated with at least one of a body massaging unit, a portable vibration applying cushion, a body support accessory, sports clothing, a waist-trimming massage belt and a treadmill.

9. The apparatus according to claim 1, wherein said modular component is disposable.

10. The apparatus according to claim 1, wherein said modular component also includes at least one carrier having on one surface a pattern of a plurality of spaced apart applying elements operative to apply to the body at least one type of energy selected from a group consisting of RF energy, light energy and mechanical energy.

11. The apparatus according to claim 1, wherein said RF energy applied to the body by said RF delivery electrode is in the range of 1 W-40 W in accordance with the type of applied treatment and part of body to be treated.

12. The apparatus according to claim 1, wherein said apparatus controller also includes at least one of an automatic and manual input functionality that enables entering of information relating to user information, preferences and settings and data received from previous treatment sessions.

13. The apparatus according to claim 12, wherein said automatic input functionality enables automatic entering of information received from at least one of a sensor and timer.

14. The apparatus according to claim 1, wherein said controller is also operative to carry out at least one of the following functionalities:
adjust cosmetic treatment parameters in accordance with said predetermined treatment protocol;
adjust cosmetic treatment parameters in relation to information input from at least one of said sensor and timer;
cease and resume treatment application in accordance with at least one of predetermined treatment parameters and duration of treatment;
alert a user regarding a deviation from said treatment parameters in accordance with predetermined treatment protocol; and
provide a user with a disposable modular component replacement time alert.

15. The apparatus according to claim 1, wherein said controller is also operative to:
limit duration of treatment to avoid overheating of tissue;
limit use time of said energy delivery modular component;
provide a disposable modular component replacement time alert, following a predetermined number of uses or maximal allowable duration of use; and
provide said controller with usage data such as number of uses, duration of use and dates of use.

16. The apparatus according to claim 1, wherein said controller is also operative to communicate with the internet, receive information therefrom and adjust cosmetic treatment parameters in accordance with said received information.

17. The apparatus according to claim 1, wherein said source of RF energy is at least one of a disposable source of energy and a rechargeable source of energy.

18. A disposable energy delivery modular component for a body contouring apparatus, said component comprising:
at least one body accommodating contact surface operative to accommodate at least part of a subject's body;
at least two RF energy delivery electrodes mounted on said contact surface, operative to apply RF energy to said at least part of said subject's body and generate a sweeping tissue heating wave effect creating a progression of a heated zone across a segment of tissue to be treated;
at least one mechanical energy delivery element operative to transfer mechanical energy to said at least part of said subject's body; and
at least one sensor.

19. A method of a cosmetic treatment, said method comprising:
coupling to a segment of body a disposable energy delivery component;
employing said energy delivery component to apply at least one type of energy and generate a sweeping tissue heating wave effect creating a progression of a heated zone across a segment of tissue to be treated;
sensing at least one of the effects caused by at least one of the energies and providing feedback; and
modifying at least one type of said energies applied to a segment of the body and treatment parameters in accordance with said feedback and a predetermined treatment protocol.

20. The method according to claim 19, wherein said effect is at least one of linear and rotational sweeping tissue heating wave effect.

21. The method according to claim 19, wherein said types of energy are selected from a group consisting of RF energy, light energy and mechanical energy.

22. The method according to claim 19, wherein said RF energy applied to the body is in the range of 1 W-40 W in accordance with the type of applied treatment and part of body to be treated.

23. The method according to claim 19, wherein said feedback is at least one type of feedback selected from a group consisting of temperature, pressure, and impedance.

24. The method according to claim 19, wherein said treatment parameters are at least one parameter selected from a group consisting of temperature, pressure and RF voltage and frequency.

25. The method according to claim 19, wherein also applying vibration energy.

26. The method according to claim 19, wherein also:
ceasing and resuming treatment application in accordance with at least one of predetermined treatment parameters and duration of treatment;
alerting a user regarding a deviation from said treatment parameters in accordance with predetermined treatment protocol;
providing a user with a disposable modular component replacement time based on a predetermined number of uses or maximal allowable duration of use;
limiting duration of treatment to avoid overheating of tissue;
limiting use time of a disposable energy delivery modular component; and
collecting and storing usage data such as number of uses, duration of use and dates of use.
